(19) United States
(12) Patent Application Publication
(10) Pub. No.: US 2018/0200285 A1
(43) Pub. Date: Jul. 19, 2018

(54) DIETARY PREBIOTIC SUPPLEMENT AND RELATED METHOD

(71) Applicant: Access Business Group International LLC, Ada, MI (US)

(72) Inventors: Brandon C. Iker, Grand Rapids, MI (US); Greg Hillebrand, Whitehall, MI (US)

(21) Appl. No.: 15/869,032

(22) Filed: Jan. 12, 2018

Related U.S. Application Data
(60) Provisional application No. 62/446,042, filed on Jan. 13, 2017.

(51) Int. Cl. A61K 31/718; A61K 31/736; A61K 9/00; A23L 33/125

(52) U.S. Cl. CPC A61K 31/718; A61K 31/736; A23V 2002/00; A23L 33/125; A61K 9/0053

(57) ABSTRACT

A composition and a method for improving the gastrointestinal (GI) health of a human subject by promoting the activity of amylolytic and short-chain fatty acid (SCFA) producing (e.g. acetogenic and/or butyrogenic) microbes in the GI system of a human subject. The composition includes a mixture of microbiota accessible carbohydrates (MACs) that includes at least two different resistant starches and at least two different non-starch polysaccharides. A blend for inclusion in a dietary supplement includes, or is, the mixture.

### CROSS-REFERENCE TO RELATED APPLICATIONS

This application claims priority to and all advantages of U.S. Provisional Patent Application No. 62/446,042 filed on 13 Jan. 2017, the contents of which is hereby incorporated by reference.

### FIELD OF THE INVENTION

The present embodiments generally relate to a composition and to a method for improving the gastrointestinal (GI) health of a human subject by promoting the activity of amylolytic and short-chain fatty acid (SCFA) producing microbes in the GI system of the human subject. The composition can include a mixture of different types of microbiota accessible carbohydrates (MACs), and more particularly a mixture of at least two different resistant starches and at least two different non-starch polysaccharides.

### DESCRIPTION OF THE RELATED ART

The human gastrointestinal (GI) system includes a variety of microbial cells forming a GI microbiota that is associated with multiple aspects of the host's health, including the health of the host's digestive system, immune system, and metabolism. A variety of factors can affect the human GI microbiota, including diet, antibiotics, and disease. Microbiota dysbiosis has been associated with a variety of diseases such as metabolic syndrome, inflammatory bowel disease, and cancer. Thus, it is believed that manipulating the GI microbiota can be used to treat and/or prevent disease.

Food ingested by the host, particularly soluble dietary fiber, is the most common fuel for GI microbiota. Microbiota accessible carbohydrates (MACs) are defined as carbohydrates that are dietary and resistant to degradation and absorption by the host, but which can be metabolically processed by GI microbes. The byproducts of GI microbe metabolism are often utilized by the host and can thus affect the health of the host. For example, soluble dietary fiber can be utilized by GI microbiota to produce short-chain fatty acids (SCFAs), such as butyrate, propionate, and acetate, which are useful to the host. Butyrate in particular is an important SCFA, as it is utilized as a primary energy source for colon cells, and thus can affect the health of the colon.

MACs can affect the composition, diversity, and function (e.g. metabolic output) of human GI microbiota. The growth of a particular species of microbe may be impacted depending on the type of MACs ingested by the host, resulting in the growth of certain microbe species over others. MACs may be metabolically processed differently or not at all depending on the species of microbe, thus affecting the availability of certain fermentation byproducts utilized by the host. Depending on the MACs ingested by the host and the composition of the host's GI microbiota, the output of a given fermentation byproduct can vary greatly among different hosts. In other words, because the carbohydrates metabolized by a host depend upon the composition or makeup of each host's microbiota, the effect of a certain MAC on a human can vary over time and can vary between different individuals.

While various efforts have been made, there remains an opportunity to provide additional methods and compositions for improving the GI health of a human subject by modulating the composition, diversity, and/or function of the human GI microbiota. In addition, there remains an opportunity to provide methods and compositions for promoting the production of desirable gut microbiota fermentation byproducts across a diverse spectrum of human subjects.

### BRIEF SUMMARY OF THE INVENTION

A method and a composition are disclosed. The method and composition are useful for improving the gastrointestinal (GI) health of a human subject by promoting activity of amylolytic and short-chain fatty acid (SCFA) producing microbes in the human GI microbiota. The composition includes a mixture of different types of microbiota accessible carbohydrates (MACs) which promotes SCFA production in the GI of a human subject through promoting the activity of amylolytic microbes. The composition can also include one or more pharmaceutically acceptable additives. The composition is formulated to provide multiple sources of MACs that stimulate activity in microbes that produce SCFAs to mitigate intra- and interpersonal variation in the gut microbiome of each human subject. In some embodiments, the composition is formulated to provide a mixture of MACs that stimulates activity in microbes that produce SCFAs in different locations within the GI system.

In one embodiment, a composition for treating the GI microbiota of a human includes a mixture of MACs and one or more pharmaceutically acceptable additives. The mixture includes at least two different resistant starches, at least two different non-starch polysaccharides. The mixture promotes the activity of amylolytic and SCFA producing microbes (e.g. acetogenic and/or butyrogenic microbes) in the GI system of a human.

In another embodiment, the resistant starches are selected from the group consisting of starchy vegetables, starch producing roots, tubers, bulbs, corms, legumes, nuts, seeds, and combinations thereof. The resistant starches can be selected from the group consisting of potato starch, pea starch, tiger nut starch, yacon starch, green plantain starch, raw green banana starch, and combinations thereof. The non-starch polysaccharides can be selected from the group consisting of mannans, arabinoglycans, arabinogalactans, rhamnans, beta-glucans, galactans, and combinations thereof.

In yet another embodiment, each of the at least two different resistant starches are present in the composition in an amount that is at least twice the amount of each of the at least two different non-starch polysaccharides.

In another embodiment, the mixture promotes the amylolytic and SCFA producing activity of at least some members of the taxa belonging to Clostridiaceae, Actinobacteria, and/or Bacteroidetes present in the colon of a human.

In still another embodiment, the at least two different resistant starches comprise at least three of pea starch, potato starch, raw green banana starch, and yacon starch. The at least two different non-starch polysaccharides comprise glucomannon and acacia gum.

In another embodiment, the one or more pharmaceutically acceptable additives is selected from the group consisting of: excipients, diluents, binders, granulating agents, glidants, flow aids, fillers, lubricants, preservatives, stabilizers, coatings, disintegrants, sweeteners, flavors, pigments, and combinations thereof.

In another embodiment, the at least two different resistant starches and the at least two different non-starch polysaccharides are present in the mixture of MACs in an amount effective to improve the health of the GI microbiota. Improving the health of the GI microbiota may include at least one of increasing a diversity of the GI microbiota, improving a composition of the GI microbiota, and/or increasing SCFA production by the GI microbiota, optionally at least one of improving a composition of the GI microbiota and/or increasing SCFA production by the GI microbiota. In various embodiments, at least SCFA production by the GI microbiota is increased.

In another embodiment, the at least two different resistant starches and the at least two different non-starch polysaccharides are present in the mixture of MACs in an amount effective to improve bowel movement health in a human.

In still another embodiment, the at least two different resistant starches and the at least two different non-starch polysaccharides are present in the mixture of MACs in an amount effective to at least one of: improve satiety, reduce systemic inflammation, increase skin microbiome diversity in at least one of facial skin and scalp skin, improve facial skin complexion, improve sleep quality, improve blood testosterone levels, improve estradiol blood levels, improve blood lipoprotein lipid levels, improve blood triglyceride levels, improve mood, improve cognition, and/or improve blood cholesterol levels.

In one embodiment, a blend for inclusion in a dietary supplement consists of at least two different resistant starches and at least two different non-starch polysaccharides. The at least two different resistant starches and at least two different non-starch polysaccharides are present in the blend in an amount effective to promote the activity of amylolytic and SCFA producing microbes in the GI system of a human.

In one embodiment, a method of increasing production of SCFAs in the GI system of a human is provided. The method includes orally administering to the human a mixture of MACs comprising at least two different resistant starches and at least two different non-starch polysaccharides. The at least two different resistant starches and the at least two different non-starch polysaccharides are present in the mixture in an amount effective to promote the activity of amylolytic and SCFA producing microbes in the GI system of the human.

Without being bound or limited by any particular theory, it is thought that the method and composition of this disclosure are useful for improving the GI health of a human subject by modulating the composition, diversity, and/or function of human GI microbiota. In addition, the method and composition of this disclosure are useful for promoting the activity of bacteria which produce SCFAs. Moreover, it is thought that the method and composition of this disclosure are useful for promoting the activity of bacteria that are amylolytic and SCFA producing (e.g. acetogenic and/or butyrogenic bacteria). Still further, it is thought that the method and composition of this disclosure are useful for promoting the activity of anaerobic fermenters in the colon of a human subject, such as the taxa belonging to Clostridiaceae, Actinobacteria, and/or Bacteroidetes. In addition, the method and composition provides multiple sources of MACs that stimulate activity in microbes that produce SCFAs to mitigate intra- and interpersonal variation in the GI microbiome of each human subject. Still further, the combination of the at least two non-starch polysaccharides with the at least two resistant starches promotes SCFA production in microbes located in different parts of the GI system than the microbes which metabolize resistant starches.

It is thought that improving GI health according to the method and composition of this disclosure may be associated with improving the health of one or more additional aspects of the human subject. For example, the method and composition may improve the health of the GI microbiota. Improving the health of the GI microbiota can include at increasing a diversity of the GI microbiota, improving a composition of the GI microbiota, and/or increasing SCFA production by the GI microbiota. In another example, the method and composition of this disclosure may be associated with improving bowel movement health in a human, such as by improving the quality, frequency, and/or comfort of bowel movements. In still another example, the method and composition of this disclosure may be associated with improvement in one or more additional aspects of health, such as improving satiety, reducing systemic inflammation, increasing a skin microbiome diversity in facial and/or scalp skin, improving facial skin complexion, improving sleep quality, improving blood testosterone levels, improving estradiol blood levels, improving blood lipoprotein lipid levels, improving blood triglyceride levels, improving mood, improving cognition, and/or improving blood cholesterol levels. In various embodiments, at least one of the following effects is achieved: increasing satiety (e.g. decreasing appetite), promoting weight loss, reducing systemic inflammation, improving facial skin complexion, increasing blood testosterone levels, improving blood lipoprotein lipid levels, decreasing blood triglyceride levels, improving mood, improving cognition, and/or decreasing blood cholesterol levels.

These and other objects, advantages, and features of the invention will be more fully understood and appreciated by reference to the description of the current embodiment and the drawings. Before the embodiments of the present invention are explained in detail, it is to be understood that the invention is not limited to the details of operation or to the details of construction and the arrangement of the steps or components set forth in the following description or illustrated in the drawings.

Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of steps or components. Nor should the use of enumeration be construed as excluding from the scope of the invention any additional steps or components that might be combined with or into the enumerated steps or components.

### BRIEF DESCRIPTION OF THE DRAWINGS

Other advantages of the disclosure will be readily appreciated, as the same becomes better understood by reference to the following detailed description when considered in connection with the accompanying drawings wherein:

FIG. 1 is a schematic overview of the experimental design of a randomized, 2-period crossover study completed to evaluate the effects of the composition of Example 1 on the human microbiome and associated parameters.

### DETAILED DESCRIPTION OF THE INVENTION

A composition, a blend, and a method are disclosed. The composition, blend, and method are useful for improving the gastrointestinal (GI) health of a human subject by promoting the activity of amylolytic microbes that produce short-chain fatty acids (SCFAs). In various embodiments, the method, blend, and composition promote the activity of microbes that are amylolytic and acetogenic and/or butyrogenic. The method, blend, and composition are also useful for promoting the activity of anaerobic fermenters of the taxa belonging to Clostridiaceae, Actinobacteria, and/or Bacteroidetes in the GI of a human subject, particularly in the colon of the human subject. The composition, blend, and method may also mitigate intra- and interpersonal variation in the GI microbiome of each human subject by providing multiple sources of microbiota accessible carbohydrates (MACs) that stimulate activity in microbes that produce SCFAs. The composition, blend, and method may also promote amylolytic and SCFA-producing microbes in multiple locations within the GI system. The composition, blend, and method may also decrease the intestinal permeability associated with a leaky gut. Other potential non-limiting benefits are described herein.

Each of the composition and blend includes a mixture of different types of MACs. In various embodiments, the MAC mixture includes at least two different resistant starches and at least two different non-starch polysaccharides. As used herein, the phrase “microbiota accessible carbohydrates,” also referred to as “MACs,” encompasses carbohydrates that can be consumed, but are resistant to digestion by a human subject, and which can be metabolically utilized by gut microbes in the human subject. As used herein, the phrase “resistant starch” encompasses starch macromolecules and/or starch degradation products that are resistant to digestion by the host, but are metabolized through fermentation by human gut microbiota. In various embodiments, the blend for inclusion in a dietary supplement includes, or is, the MAC mixture.

In certain embodiments, the MAC mixture consists essentially of at least two different resistant starches and at least two different non-starch polysaccharides. As used herein, the phrase “consisting essentially of” generally encompasses the specifically recited elements/components for a particular embodiment. Further, the phrase “consisting essentially of” generally encompasses and allows for the presence of additional or optional elements/components that do not materially impact the basic and/or novel characteristics of that particular embodiment. In certain embodiments, “consisting essentially of” allows for the presence of ≤10, ≤5, or ≤1, percent by weight (% wt.) of additional or optional components based on the total weight of the composition.

In various embodiments, the composition comprising the MAC mixture including at least two different resistant starches and at least two different non-starch polysaccharides additionally includes pharmaceutically acceptable additives that are inactive ingredients as described below. If utilized, the inactive ingredients are different from the resistant starches and non-starch polysaccharides of the MAC mixture (and optional other active ingredients).

Examples of primarily inactive ingredients include, but are not limited to, flavorings; carob; alkalized, organic, or other forms of cocoa; natural chocolate flavor, natural vanilla flavor, or other natural flavorings; coconut sugar; pea protein or other sources of dietary protein; sea salt; stevia powder or other sweeteners; corn syrups, such as hydrolyzed corn syrup solids; modified cellulose, methyl cellulose, hydroxypropyl methyl cellulose, and carboxy methyl cellulose; maltodextrin and maltol, such as natural maltol; fructose; sorbitol; preservatives; alcohols, such as ethanol, propyl alcohol and benzyl alcohol; glycerin; potassium sorbate; sodium benzoate; binders; flow agents; stearates, such as calcium stearate, magnesium stearate, and sodium magnesium stearate; dicalcium phosphate; glyceryl triacetate; vegetable oils, such as hydrogenated vegetable oils; mineral oils; water; silicones, such as silicone oils; silicon dioxide; stearic acid; waxes, such as carnauba wax and beeswax; fatty esters and fatty alcohols; glycols and polyglycols; and combinations thereof. If utilized to form the composition, the inactive ingredient(s) can be used in various amounts and combined with the resistant starches and non-starch polysaccharides of the MAC mixture to form the composition. Further, it is to be appreciated that the amounts of actives described herein can be normalized with respect to 100 parts by weight of the composition to account for the presence of inactive ingredients (if utilized).

Regarding the resistant starches of the MAC mixture, the resistant starch may be any suitable type of resistant starch from a source that is generally considered safe for human consumption. In various embodiments, the MAC mixture includes one or more types of resistant starches that are metabolized by the GI microbiota to generate SCFAs. In certain embodiments, the resistant starches in the MAC mixture include a physically inaccessible starch (RS1 type), a crystalline resistant starch granule (RS2 type), or combinations thereof. Non-limiting examples of sources of RS1 type starches include whole or partly milled grains, seeds, and legumes. Non-limiting examples of sources of RS2 types starches include raw potato, sweet potato, legumes, plantains, and high amylose maize. Depending on the source and processing method, a particular resistant starch source may provide both RS1 and RS2 types of starches.

In various embodiments, the RS1 starch component is selected from starchy vegetables, whole grains, legumes, dried and ground roots, tubers, corms, bulbs, nuts, seeds, tiger nuts, sweet potato starch, cattail root starch, cattail rhizome starch, cassava starch, arrow root starch, palm starch, chick pea starch, and combinations thereof. In certain embodiments, the MAC mixture includes at least two different types of RS1 starches.

In various embodiments, the RS2 type starch component is selected from raw unprocessed or native potato starch, raw unprocessed tiger nut flour, raw unprocessed green banana flour, raw unprocessed or native pea starch, raw unprocessed or native yacon starch/flour, green plantain starch, sweet potato starch, cattail root starch, cattail rhizome starch, cassava starch, arrow root starch, palm starch, chick pea starch, or combinations thereof. In certain embodiments, the MAC mixture includes at least two different types of RS2 starches.

In various embodiments, the resistant starch is obtained from a plant source using minimal processing and cooking to preserve the structure of the starch and optionally to preserve the structure of the amyloplast. Starch obtained from raw plant sources generally has a higher amount of resistant starch than starch obtained from heated or cooked plant source. However, the processing and cooking techniques used in obtaining the resistant starch may vary depending on the desired level of preservation of the amyloplasts and/or starch granules.

In various embodiments, the source of the resistant starch may be selected based on the morphology of the starch granules. In certain embodiments, the source of resistant starch is selected to provide A-type and/or B-type starch granules, which are resistant to digestion. The presence of A-type and/or B-type starch granules can be determined according to traditional methods using polarized light. One or more of the resistant starches can be selected to provide the desired amount of A-type and/or B-type starch granules in the MAC mixture. The resistant starch can be commercially purchased in a form suitable for human consumption.

Regarding the non-starch polysaccharides of the MAC mixture, the non-starch polysaccharides may be any suitable polysaccharide that promotes the activity of gut microbes that generate SCFAs. In certain embodiments, the non-starch polysaccharides are selected to promote the activity of SCFA generating microbes in areas of the GI system different than that of the resistant starches. In specific embodiments, the non-starch polysaccharides are selected to promote the activity of gut microbes that generate butyrate. Non-limiting examples of non-starch polysaccharides suitable for use in the MAC mixture include mannans, glucomannan, arabinoglycans, arabinogalactans, galactans, rhamnans, galactans, guar gum, beta-glucans, xanthan gum, acacia or acacia gum, and combinations thereof. The non-starch polysaccharides can be commercially purchased in a form suitable for human consumption. Examples of commercially available guar gum and acacia products include Sunfiber® and Fibregum™ respectively, both of which are commercially available from multiple sources.

The MAC mixture includes at least two different resistant starches, optionally at least three different resistant starches, optionally at least four different resistant starches. In further embodiments, the MAC mixture (or the composition as a whole) includes just two different resistant starches, optionally just three different resistant starches, optionally just four different resistant starches. In alternate embodiments, the MAC mixture (or the composition as a whole) includes at least one or just one resistant starch.

The MAC mixture further includes at least two different non-starch polysaccharides, optionally at least three different non-starch polysaccharides. In further embodiments, the MAC mixture (of the composition as a whole) includes just two different non-starch polysaccharides, optionally just three different non-starch polysaccharides. In alternate embodiments, the MAC mixture (or the composition as a whole) includes at least one or just one non-starch polysaccharides. In other alternate embodiments, the MAC mixture (or the composition as a whole) is free of non-starch polysaccharides.

In certain embodiments, the MAC mixture includes three different resistant starches and three different non-starch polysaccharides. In other embodiments, the MAC mixture includes three different resistant starches and two different non-starch polysaccharides. In yet other embodiments, the MAC mixture includes four different resistant starches and two different non-starch polysaccharides. The at least two different resistant starches may differ from each other based on source, chemical structure, and/or physical structure. The at least two different non-starch polysaccharides may differ from each other based on source, chemical structure, and/or physical structure.

In various embodiments, the at least two different resistant starches comprise at least two of, optionally at least three of, optionally all four of, pea starch, potato starch, raw green banana starch, and yacon starch. In these and other embodiments, the at least two different non-starch polysaccharides comprise glucomannon and acacia gum.

In various embodiments, the at least two different resistant starches can each individually be present in the composition in an amount of from about 1 to about 95 percent by weight (% wt.), optionally about 5 to about 90% wt., optionally about 10 to about 80% wt., optionally about 10 to about 50% wt., optionally about 15 to about 35% wt., optionally about 15 to about 20% wt., optionally about 16 to about 19% wt., optionally about 18% wt. Each of the different resistant starches can be present in the composition in the same or differing amounts, e.g. one resistant starch is present in an amount of about 20% wt. and another resistant starch is present in an amount of about 12% wt., each of three different resistant starches are individually present in an amount of about 18% wt., etc. The blend can include the same amounts, or higher, normalized amounts to account for the absence of pharmaceutically acceptable additives.

In certain embodiments, the at least two different resistant starches are present in the composition such that a total amount of resistant starch consumed by the human subject is an amount of from about 1 to about 250 grams per day, optionally about 5 to about 200 grams per day, optionally about 10 to about 150 grams per day, optionally about 15 to about 100 grams per day. The composition may be formulated to provide the desired total amount of resistant starch in a single dose or multiple doses. In certain embodiments, the at least two different resistant starches can each be present in the MAC mixture in an amount of from about 1 to about 10 grams per dose, optionally about 2 to about 8 grams per dose, optionally about 3 to about 6 grams per dose, optionally about 4 grams per dose. The total number of doses consumed by the human subject can vary based on the desired total amount of resistant starch to consume per day. The resistant starches may each be present in the same or different amounts. It is contemplated that any and all values or ranges of values between those described above may also be utilized.

In various embodiments, the at least two different non-starch polysaccharides can each individually be present in the composition in an amount of from about 0.1 to about 25% wt., optionally about 0.5 to about 15% wt., optionally about 1 to about 10% wt., optionally about 1 to about 5% wt. The different non-starch polysaccharides can each be present in the composition in the same or differing amounts, e.g. one non-starch polysaccharide is present in an amount of about 10% wt. and another non-starch polysaccharide is present in an amount of about 5% wt., each of two different non-starch polysaccharides are individually present in an amount of about 8% wt., etc. The blend can include the same amounts, or higher, normalized amounts to account for the absence of pharmaceutically acceptable additives.

In certain embodiments, the at least two different non-starch polysaccharides are present in the composition such that a total amount of non-starch polysaccharides consumed by the human subject is an amount of from about 1 to about 100 grams per day, optionally about 1 to about 75 grams per day, optionally about 1 to about 50 grams per day, optionally about 1 to about 25 grams per day. The composition may be formulated to provide the desired total amount of resistant starch in a single dose or multiple doses. In certain embodiments, the at least two different non-starch polysaccharides can each be present in the in the MAC mixture in an amount of from about 0.05 to about 10 grams per dose, optionally about 0.1 to about 7.5 grams per dose, optionally about 0.25 to about 5 grams per dose, optionally about 0.5 to about 3 grams per dose, optionally about 1 to about 2 grams per dose. The total number of doses consumed by the human subject can vary based on the desired total amount of non-starch polysaccharides to consume per day. The non-starch polysaccharides may each be present in the same or different amounts. It is contemplated that any and all values or ranges of values between those described above may also be utilized.

In various embodiments, the total amount of resistant starches and the total amount of non-starch polysaccharides can be present in the MAC mixture in a weight ratio of resistant starches:non-starch polysaccharides of about 6:1, optionally about 5:1, optionally about 4:1. In other words, the total amount of resistant starches may be present in an amount of about 4 to about 6 times more than an amount of the non-starch polysaccharides.

In various embodiments, the amount of each individual resistant starch and each individual non-starch polysaccharide is present in the MAC mixture in a weight ratio of resistant starch:non-starch polysaccharides of about 4:1, optionally about 2:1. In other words, each individual resistant starch may be present in an amount that is about 2 to about 4 times more than an amount of each individual non-starch polysaccharide.

In various embodiments, the total amount of resistant starches present in the composition is about 10 to about 90% wt. of the MAC mixture. In further embodiments, the total amount of non-starch polysaccharides present in the composition is about 10 to about 90% wt. of the MAC mixture. It is contemplated that any and all values or ranges of values between those described above may also be utilized.

Surprisingly, it was discovered that a greater amount of the at least two different resistant starches than the at least two different non-starch polysaccharides provides unexpected benefits to the human subject. There is wide intra- and interpersonal variation in the human microbiome in terms of both the composition and population of microbes present. An individual's microbiome may vary depending on diet, medication, and other health issues. In addition, the microbiome of one individual may vary significantly from another's, resulting in a different physiological response to the same treatment. Still further, some MACs, such as the non-starch polysaccharide guar gum, are metabolized and produce SCFAs in locations within the GI system that are different than where resistant starches are metabolized. It is contemplated that the combination of at least two different resistant starches and at least two different non-starch polysaccharides in the present combinations/mixtures mitigate the intra- and interpersonal variation in human GI microbiome by providing a diverse mixture of resistant starches and other fermentable carbohydrates. It is also contemplated that the combination of at least two different resistant starches and at least two different non-starch polysaccharides in the present combinations/mixtures address variations in the location within the GI system of amylolytic and SCFA-producing microbiota.

Specifically, it was discovered that the combinations/mixtures described herein modulate the composition, diversity, and/or function of the GI microbiota in the human subject. Modulating the GI microbiota may provide direct beneficial effects on the GI health of a human subject in addition to other aspects of human health. Additional beneficial effects include promoting the production of SCFAs, and in particular in promoting the production of at least one of butyrate, propionate, and/or acetate, optionally at least butyrate, optionally at least acetate, optionally at least acetate and butyrate. Additional beneficial effects include promoting the activity of at least some anaerobic fermenters present in the human colon, examples of which include members of the taxa belonging to Clostridiaceae, Actinobacteria, and/or Bacteroidetes. These anaerobic fermenters may improve production of SCFAs in the colon. Still further benefits may be seen in changes in skin and oral microbiota, facial skin features, production of GI microbiota metabolites, changes in sleep outcomes, changes in appetite sensations, changes in bowel habits, GI quality and tolerance, heart rate variance, fasting lipoprotein lipids, and/or blood markers associated with GI permeability and systemic inflammation. It is also thought that the combinations/mixtures herein decrease the intestinal permeability associated with a leaky gut. Without being bound or limited to a particular theory, it is thought that the combinations/mixtures herein help to boost a person's gut barrier.

The composition can be prepared using various methods. For example, actives of the composition, and optionally one or more inactives, can be mixed or blended and compressed or compounded utilizing various techniques understood in the art. The composition of this disclosure is not limited to a particular order of manufacturing steps or method of manufacture.

Typically, the composition is administered (or ingested) orally, e.g. via the mouth (or “per os”). More typically, at least a portion of the composition is administered (or digested) enternally, e.g. via the GI track (or “enteros”). The subject is typically a human, and can include men and women of various ages. The method/composition of this disclosure is not limited to a particular subject.

The composition can be in various forms. Examples of suitable forms include solids, gels, and liquids. In various embodiments, the composition can be solid. For example, the composition can be in the form of a pill, including tablets, capsules, and caplets. In general, each of these terms can be used interchangeable in the art, e.g. tablet for pill or vice versa. Further form examples include those that mimic conventional foods and/or drinks, such as bars, shakes, gummies, sachets, cereals, etc.

Other than the MACs (i.e., the “actives” or “active ingredients”), the composition can include pharmaceutically acceptable additives that are inactive (or “inactive ingredients”) including, but not limited to, excipients, such as diluents and binders; granulating agents; glidants (or flow aids); fillers; lubricants; preservatives; stabilizers; coatings; disintegrants; sweeteners or flavors; and pigments. Further examples of inactive ingredients are described above. The MACs and the pharmaceutically acceptable additives can be combined or compounded as desired to form an individual dose that provides the desired amount of MACs to the human subject when consumed. Optionally, a number and quantity of excipients can be kept at a minimum as long as active ingredients are properly delivered to satisfy subjects' preference for smaller tablets that are easier to consume. In various embodiments, the MACs and the pharmaceutically acceptable additives are combined to form an individual dose that is intended to be consumed by the human subject in multiples in order to provide the desired total amount of resistant starch and non-starch polysaccharide MACs to the human subject during treatment.

Each of the composition and blend can be in powder form, or pressed or compacted from a powder into a solid dose. A coating, e.g. polymer coating, may be used to make the tablet smoother and easier to swallow, to control release rate of the actives, to increase resiliency (or shelf life), and/or to enhance appearance. Other suitable oral forms of the composition include syrups, elixirs, suspensions, and emulsions.

In general, tablets provide a solid dosage form of delivery by oral route. Typically, the main purpose of a tablet formulation is to deliver active ingredients to a subject/consumer. Inactive ingredients are inactive substances that are generally used as carriers and formulation support for delivery of active ingredients. Inactive ingredients can be used for a variety of reasons, including handling small quantities (low mg and mcg doses) of active ingredients, accurate dosing, stabilizing unstable active ingredients, degradation of active ingredients in the stomach, diluting active ingredients to prevent potential GI tract injury, and/or masking unpleasant organoleptic properties (taste and smell) of active ingredients.

The composition can be administered to a human subject in various amounts. In various embodiments, the composition can be administered such that a total amount of MACs administered to the subject is at least about 5 grams per day, optionally at least about 10 grams per day, optionally at least about 15 grams per day, optionally at least about 20 grams per day, optionally at least about 25 grams per day, optionally at least about 30 grams per day, optionally at least about 40 grams per day, optionally at least about 45 grams per day. In certain embodiments, the total amount of MACs is divided among multiple doses of about 5 grams each, optionally about 10 grams each, optionally about 15 grams each. The number of doses and the amount of MACs per dose can vary based on the human subject's tolerances and the desired outcome. For example, the number of doses and the amount of MACs per dose may be selected to provide a minimum amount or increase of SCFAs in the human subject's feces.

The composition may be administered as needed, daily, several times per day or in any suitable regimen such that the desired outcome is achieved. In the method of this disclosure, the frequency of administration (e.g. of ingestion and/or digestion) can depend on several factors, including the desired level of SCFA production and GI tolerance. Generally, a regimen includes administration of the composition once or twice daily to include an administration in the morning and/or an administration in the evening. In certain embodiments, the regimen can include administration three times daily in the morning, afternoon, and evening. The amount of composition administered may depend on several factors, including level of desired results and the specific composition.

In various embodiments, the composition is administered to the subject on a periodic basis, optionally on a daily basis, as part of a nutritional supplement regime for improving GI health in a human subject. In these embodiments, the MAC mixture may be provided as a powder that is mixed with a liquid, such as water, to form a beverage for consumption. The beverage may be consumed at or in between meal times. In various embodiments, the MAC mixture may be mixed with a food, an example of which includes yogurt or oatmeal, and consumed by the human subject.

The following examples, illustrating the compositions and methods of this disclosure, are intended to illustrate and not to limit the disclosure.

### EXAMPLES

### Example 1

A MAC mixture including three different resistant starches and three different non-starch polysaccharides was blended with additional inactive ingredients to form a dry chocolate shake mix composition (“Shake Mix A”). The Shake Mix A composition including sweeteners, flavorings, and other inactive ingredients is formulated to facilitate mixing the composition with water to form a chocolate shake-tasting beverage for consumption by a human subject. Table 1 below identifies the components and their relative amounts in Shake Mix A. Shake Mix A can be formulated to provide a total of 15 grams of MACs per dose, with a dose corresponding to a single scoop of powder using a measuring utensil provided with the composition. Shake Mix A may be consumed by a human subject once, or more than once a day, depending on several factors, such as the desired level of SCFA production or GI tolerance.

TABLE 1
Shake Mix A

| Component | Amount (% wt.) |
|---|---|
| Pea starch, native | 18.1 |
| Potato starch, native | 18.1 |
| Raw green banana flour | 18.1 |
| Glucomannan | 4.5 |
| Acacia gum | 4.5 |
| Guar gum, partially hydrolyzed | 4.5 |
| Coconut sugar | 7.7 |
| Pea protein | 7.9 |
| Cocoa, alkalized | 6.8 |
| Natural chocolate flavor | 4.5 |
| Natural vanilla flavor | 2.3 |
| Acacia/Xanthan gum blend | 2.3 |
| Sea salt, purified | 0.5 |
| Stevia powder, 95% organic | 0.3 |
| Total | 100 |

### Example 2

A MAC mixture including four different resistant starches and two different non-starch polysaccharides was blended with additional inactive ingredients to form a dry chocolate shake mix composition (“Shake Mix B”). The Shake Mix B composition including sweeteners and flavorings is formulated to facilitate mixing the composition with water to form a chocolate shake-tasting beverage for consumption by a human subject. Table 2 below identifies the components and their relative amounts in Shake Mix B. Shake Mix B can be formulated to provide a total of 19 grams of MACs per dose, with a dose corresponding to a single scoop of powder using a measuring utensil provided with the composition. Shake Mix B may be consumed by a human subject once, or more than once a day, depending on several factors, such as the desired level of SCFA production or GI tolerance.

TABLE 2
Shake Mix B

| Component | Amount (% wt.) | Amount (grams) |
|---|---|---|
| Pea starch, native | 16 | 4 |
| Potato starch, native | 16 | 4 |
| Raw green banana flour | 16 | 4 |
| Yacon flour | 16 | 4 |
| Glucomannan | 8 | 2 |
| Fibregum ™ (acacia gum) | 4 | 1 |
| Trehalose | 12 | 3 |
| Flavoring (organic cocoa) | 12 | 3 |
| Total | 100 | 25 |

The compositions including the MAC mixture described herein may be used to improve the health of the GI microbiota of a human subject by modulating the composition, diversity, and/or the function of the GI microbiota in the human subject. The MAC mixture can provide beneficial effects on the GI health of a human subject as well as on other aspects of human health, examples of which include the human subject's immune system and metabolism. For example, compositions and blends including the MAC mixture may decrease the intestinal permeability associated with a leaky gut.

Changes over time in the GI microbiota of treated human subjects can be determined by assessing fecal microbiota using the Shannon Diversity Index (SDI). Optionally, changes over time in the GI microbiota of treated human subjects can be determined by assessing changes in the relative abundance of amylolytic and SCFA producing (e.g. acetogenic and/or butyrogenic) microbiota.

In one example, the MAC mixture may promote the production of SCFAs by the GI microbiota. Changes in SCFA production can be measured by analyzing the amount of SCFA present in the subject's feces.

In another example, the MAC mixture may affect the fecal, skin, and/or oral microbial composition of the human subject. Such changes can be assessed using taxonomic profiling by 16S ribosomal RNA gene amplicon sequencing. Skin and oral microbiota can optionally be assessed using the Shannon Diversity index. Samples may be collected from a subject's forehead skin, scalp skin, and/or cheek skin.

In another example, the MAC mixture may affect the subject's appearance over time. For example, the subject may experience changes in facial features, such as wrinkles, texture, pores, pigment spots, redness, and/or porphyrins. Effects on the subject's appearance may be determined according to subject answers to a beauty questionnaire. Optionally, the effect on a subject's appearance is assessed using facial imaging followed by computer-assisted image analysis.

In another example, the MAC mixture may affect the subject's GI health, such as the frequency and type of bowel movements and the ease of passing stool. GI health may be also be assessed according to the amount and/or frequency of certain GI events, such as abdominal distention/bloating, gas/flatulence, borborygmus/stomach rumbling, and abdominal cramping. GI health may also be assessed based on a subject's appetite ratings. For example, the MAC mixture may have beneficial effects on a subject's feeling of satiety. An additional benefit may include promoting weight loss in the subject.

In another example, the effects of the MAC mixture on the subject's health may be assessed based on the presence of certain biochemical markers and/or changes in certain biochemical markers in the subject's blood. Non-limiting biochemical markers that may be assessed include fasting inflammatory markers (e.g. C-reactive protein, interleukin-6, interleukin-10, TNF-alpha), lipopolysaccharides, fasting lipoprotein lipids, fasting testosterone, and fasting estradiol. Optionally, effects of the MAC mixture may also be assessed according to changes in a subject's blood chemistry profile. Non-limiting examples of blood chemistry markers include albumin, alkaline phosphatase, total bilirubin, direct bilirubin, calcium, chloride, creatine, blood urea nitrogen, potassium, aspartate aminotransferase, alanine aminotransferase, sodium, total protein, carbon dioxide, osmolality, and glucose. Optionally, a blood hematology profile may be performed to assess white blood cell count, red blood cell count, hemoglobin concentration, hematocrit, mean cell volume, mean cell hemoglobin, mean cell hemoglobin concentration, neutrophils, lymphocytes, monocytes, eosinophils, basophils, and platelet count.

In another example, the effects of the MAC mixture on a subject's sleep habits, such as the average hours of sleep per night, can also be assessed.

In another example, the effects of the MAC mixture on a subject's cardiovascular health can also be assessed. For example, the effects of the MAC mixture on a subject's heart rate variance can be assessed. Changes in heart rate variance can be indicative of changes in systemic inflammation.

### Example 3

A randomized, controlled, crossover study was completed to demonstrate whether the mixture of Example 1, consumed daily for eight weeks, modulates the composition, diversity, and/or function of the human GI microbiota. Secondary objectives were to examine any secondary changes observed in skin and oral microbiota, facial skin features, production of GI microbiota metabolites, as well as specific health parameters, described in more detail below.

The study included 50% men and 50% women, 40 to 60 years of age (inclusive), with a waist circumference of greater than 40 inches (inclusive) in men or greater than 35 inches (inclusive) in women.

A control group received no treatment and an active group received three daily servings of the mixture of Example 1 providing 15 g of MACs per 1 scoop serving. The mixture, which was in powder form, was mixed with 6 to 8 ounces of water prior to being consumed by the active group individuals. All active group subjects underwent a 5-day dose-escalation period with the first dose of the escalation period beginning at Visit 2 (week 0), as described in more detail below.

The dose escalation regimen is as follows:

Day 1=1 scoop of product in the morning;
Day 2=1 scoop of product in the morning;
Day 3=1 scoop of product in the morning and 1 scoop of product in the evening;
Day 4=1 scoop of product in the morning and 1 scoop of product in the evening; and
Day 5=1 scoop of product in the morning, 1 scoop of product in the afternoon, and 1 scoop of product in the evening.

In cases where a subject is determined to suffer adverse side-effects, a Clinical Investigator will determine a highest tolerable dose for the subject and the subject will then remain on that highest tolerable dose for the duration of the active test period.

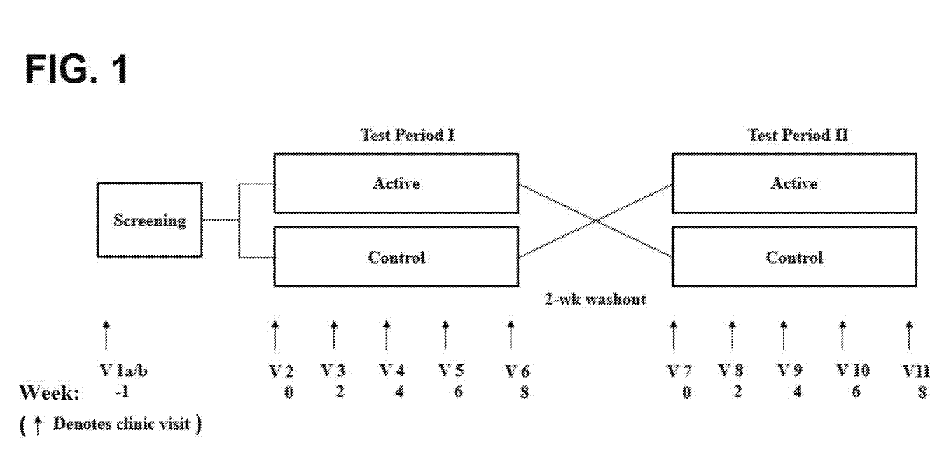

The study was a randomized, 2-period crossover study. Subjects attended one screening visit (Visit 1a/b, week-1) and, if eligible, were randomly assigned to one of two sequences, as shown in FIG. 1 (Active→4 Control or Control→4 Active). Subjects participated in a total of ten visits to a clinic for evaluation over two separate 8-week test periods [Visits 2, 3, 4, 5, and 6; weeks 0, 2, 4, 6, and 8 (Period I); Visits 7, 8, 9, 10, and 11; weeks 0, 2, 4, 6, and 8 (Period II)]. The two test periods are separated by a 2-week washout where subjects do not consume the mixture of Example 1.

At visit 1a (week −1), after providing informed consent, subjects underwent evaluation of waist circumference to identify individuals with a waist circumference 02 cm (40 inches) in men or ≥89 cm (35 inches) in women, as an initial inclusion criterion, as stated above.

Eligible subjects continued to visit 1b (week −1), to undergo the remaining screening visit procedures including, evaluations of medical history, inclusion and exclusion criteria, prior and current medication/supplement use, height, body weight, vital signs, and a last menses query, where appropriate. The inclusion and exclusion criteria, including waste size and age as introduced above, are so defined so as to target a population of interest and to exclude factors likely to have a biasing or otherwise undesirable influence on study outcomes, such as, for example, a subject's inability or unwillingness to properly participate in the study, drug use or taking of certain medications, or a medical history reflecting poor GI health. A fasting (12±2 h; water only) chemistry profile and hematology panel, and an in-clinic urine pregnancy test (all women) were obtained. Subjects were then dispensed a Bowel Habits Diary to complete over the 3-day (3-d) period immediately prior to Visit 2 (week 0). Additionally, a fecal sample collection kit was dispensed with instructions to collect feces (one bowel movement) occurring during that same 3-d period subjects are keeping the Bowel Habits Diary. If a subject was not able to produce a fecal sample during this period, the visit was rescheduled until a fecal sample could be provided. A 3-d Diet Record with instructions to record intake during the same 3-d period was also dispensed.

At Visit 2 (week 0), subjects arrived at the clinic fasted (12±2 h; water only), to undergo clinic visit procedures (review of inclusion/exclusion criteria, concomitant medication/supplement use, body weight, vital signs assessment, and a last menses query, where applicable). Adverse events (AE) were assessed and fasting blood samples were collected for analysis of testosterone (free and total) and estradiol, lipids, inflammatory markers, and lipopolysaccharide concentrations. Additional blood samples were collected for backup and archived for possible future analysis of non-genetic indicators of metabolism. A processed fecal sample was collected and the 3-d Diet Record and Bowel Habits Diary were collected/reviewed. Subjects then underwent collection of forehead skin, scalp skin, and buccal microbiome samplings, facial imaging, and heart rate variance testing. Additionally, an electronic Appetite Questionnaire and a Sleep Questionnaire were administered. Subjects were then dispensed a Bowel Habits Diary to complete over the 3-d period immediately prior to Visit 3 (week 2). Additionally, a fecal sample collection kit was dispensed with instructions to collect feces (one bowel movement) occurring during that same 3-d period. If a subject was not able to produce a fecal sample during this period, the visit was rescheduled until a fecal sample could be provided. A 3-d Diet Record with instructions to record intake during the same 3-d period was also dispensed. A wearable activity tracker was dispensed with instructions to wear the tracker 24 h/d, including sleep time. Subjects were also dispensed a GI Tolerability Questionnaire which captured any GI events over each 24-h period for the subsequent 14-d period. Subjects were then randomized to one of two sequences (Active Control or Control Active). Subjects in the active condition were then dispensed study product, with the first dose mixed and consumed in the clinic. Subjects were instructed to follow the 5-day (5-d) dose-escalation period detailed above with the dose escalating to three servings daily (morning, afternoon, and evening at approximately the same times each day). Subjects were dispensed a Study Product Diary to record study product intake for compliance assessment.

At Visit 3 (week 2), subjects arrived at the clinic fasted (12±2 h; water only) to undergo clinic visit procedures (review of inclusion/exclusion criteria, concomitant medication/supplement use, body weight, vital signs assessment, and a last menses query, where applicable). AEs were assessed and a processed fecal sample was collected. Additionally, the 3-d Diet Record, Bowel Habits Diary, and GI Tolerability Questionnaire were collected/reviewed and the activity tracker data was downloaded. The Study Product Diary was collected/reviewed and compliance was assessed (active condition only). Subjects then underwent collection of forehead skin, scalp skin, and buccal microbiome samplings, and an electronic Appetite Questionnaire was administered. Subjects were then dispensed a Bowel Habits Diary to complete over the 3-d period immediately prior to Visit 4 (week 4). Additionally, a fecal sample collection kit was dispensed with instructions to collect feces (one bowel movement) occurring during that same 3-d period subjects are keeping the Bowel Habits Diary. If a subject was not able to produce a fecal sample during this period, the visit was rescheduled until a fecal sample could be provided. A 3-d Diet Record with instructions to record intake during the same 3-d period was also dispensed. Study product and a Study Product Diary were dispensed to those in the active condition.

At Visit 4 (week 4), subjects arrived at the clinic fasted (12±2 h; water only), to undergo clinic visit procedures (review of inclusion/exclusion criteria, concomitant medication/supplement use, body weight, vital signs assessment, and a last menses query, where applicable). A processed fecal sample was collected. Additionally, the 3-d Diet Record and Bowel Habits Diary are collected and reviewed and the activity tracker data was downloaded. The Study Product Diary was collected/reviewed and compliance was assessed (active condition only). AEs were assessed and fasting blood samples were collected for analysis of inflammatory markers and lipopolysaccharide concentrations. Additional blood samples were collected for backup and archived for possible future analysis of non-genetic indicators of metabolism. Subjects then underwent collection of forehead skin, scalp skin, and buccal microbiome samplings, facial imaging, and heart rate variance testing. Additionally, subjects were administered an electronic Appetite Questionnaire, a GI Quality of Life Index, and a Sleep Questionnaire. Study product and the Study Product Diary were then dispensed to those in the active condition.

At Visit 5 (week 6), subjects arrived at the clinic (fasting not required) for AE assessment and the activity tracker data was downloaded. The Study Product Diary was collected/reviewed and compliance was assessed (active condition only). Subjects were then dispensed a Bowel Habits Diary to complete over the 3-d period immediately prior to Visit 6 (week 8). Additionally, a fecal sample collection kit was dispensed with instructions to collect feces (one bowel movement) occurring during that same 3-d period. If a subject was not able to produce a fecal sample during this period, the visit was rescheduled until a fecal sample could be provided. A 3-d Diet Record with instructions to record intake during the same 3-d period was also dispensed. Study product and a Study Product Diary were then dispensed to those in the active condition.

At Visit 6 (week 8), subjects arrived at the clinic fasted (12±2 h; water only) to undergo clinic visit procedures (review of inclusion/exclusion criteria, concomitant medication/supplement use, body weight, vital signs assessment, and a last menses query, where applicable). A processed fecal sample was collected. Additionally, the 3-d Diet Record and Bowel Habits Diary were collected and reviewed and the activity tracker data was downloaded. The Study Product Diary was collected/reviewed and compliance is assessed (active condition only). AEs were assessed and fasting blood samples were collected for analysis of testosterone (free and total) and estradiol, chemistry profile, hematology panel, lipids, inflammatory markers, and lipopolysaccharide concentrations. Additional blood samples were collected for backup and archived for possible future analysis of non-genetic indicators of metabolism. Subjects underwent collection of forehead skin, scalp skin, and buccal microbiome samplings, facial imaging, and heart rate variance testing. Additionally, subjects were administered an electronic Appetite Questionnaire, a GI Quality of Life Index, a Sleep Questionnaire, a Product Likeability Questionnaire, and a Beauty Quality of Life Questionnaire. Subjects were then dispensed a Bowel Habits Diary to complete over the 3-d period immediately prior to Visit 7 (week 0). Additionally, a fecal sample collection kit was dispensed with instructions to collect feces (one bowel movement) occurring during that same 3-d period subjects are keeping the Bowel Habits Diary. If a subject was not able to produce a fecal sample during this period, the visit was rescheduled until a fecal sample can be provided. A 3-d Diet Record with instructions to record intake during the same 3-d period was dispensed. The activity trackers were collected and subjects begin the two-week washout period.

At Visit 7 (week 0), subjects returned to the clinic and crossover to the next test sequence to repeat the procedures described above for the Test Period I, with the exception of the Sleep Questionnaire at Visit 7 (week 0). Additionally, a testosterone (free and total) and Estradiol, chemistry profile and hematology panel were included at Visit 7 (week 0).

Data was analysed using standard general methods in statistical analysis, which includes repeated measures analysis of variance (RMANOVA) followed by a multiple comparison post hoc analysis using, for example, a stepdown Bonferroni adjustment. There was a statistically significant increase in fecal SCFA production in active study participants over the control group, see Table 3 below.

TABLE 3
Fecal SCFAs (Acetic Acid, Propionic Acid, and Butyric Acid)
over time in response to active and control conditions

| | | |
|---|---|---|
| | Control | Active |
| Parameter | (N = 26) | (N = 26) |

Acetic Acid (mmol/kg)

| | | |
|---|---|---|
| Week 0 | 52.16 ± 4.12 | 54.24 ± 4.16 |
| Week 2 | 58.34 ± 3.86 | 62.83 ± 5.31 |
| Week 4 | 62.80 ± 3.39 | 56.67 ± 4.19 |
| *Week 8 | 50.39 ± 3.56 | 64.70 ± 4.54 |

Propionic acid (mmol/kg)

| | | |
|---|---|---|
| Week 0 | 20.41 ± 1.86 | 20.85 ± 2.02 |
| Week 2 | 21.61 ± 1.72 | 22.10 ± 2.02 |
| *Week 4 | 23.21 ± 1.44 | 20.44 ± 2.03 |
| Week 8 | 19.16 ± 1.45 | 22.68 ± 1.95 |

Butyric acid (mmol/kg)

| | | |
|---|---|---|
| Week 0 | 13.34 ± 2.21 | 12.88 ± 1.30 |
| Week 2 | 13.38 ± 1.41 | 15.62 ± 1.88 |
| Week 4 | 15.80 ± 1.24 | 12.96 ± 1.32 |
| Week 8 | 11.41 ± 1.16 | 14.76 ± 1.58 |

In Table 3 above, the rows marked “*” indicate a statistically significant difference (p-value <0.05) between the Active and Control groups, as calculated using a model generated on ranked values. N indicates the number of subjects in each group. All values in Table 3 are presented as mean±SEM (standard error of the mean).

A second analysis of the data was completed only for those subjects showing an increase of at least 20% over baseline (“Responder” population) for either acetate production (“A” group) or butyrate production (“B” group). These two groups of subjects showed the following statistically significant (p≤0.11) secondary outcomes, as reported in Table 4 below:

1. Tumor necrosis factor (TNF) alpha declined;
2. Skin red spots declined;
3. Appetite and urge to snack declined;
4. LDL cholesterol, non-HDL cholesterol, and total cholesterol declined;
5. Fecal consistency and ease of stool passage were improved; and
6. GI gas increased but cramping and bloating showed no significant effect

TABLE 4
Principal secondary outcomes for the Acetate (A) and
Butyrate (B) Responder sub-groups

| | | Significance | Week | |
|---|---|---|---|---|
| Parameter | Group | (p-value) | No. | Direction |
| Oral SDI | A | S | 2 | A < C |
| | B | ns | | |
| TNF-alpha | A | 0.007 | 8 | A < C |
| | B | ns | | |
| Acetate | A | 0.10 | 8 | A > C |
| | B | 0.05 | 8 | A > C |
| Propionic | A | ns | | |
| | B | 0.09 | | A > C |
| Red Spots | A | 0.059 | 4 | A < C |
| | B | 0.03 | 4 | A < C |
| LDL Cholesterol | A | 0.04 | 8 | A < C |
| | B | 0.06 | 8 | A < C |
| Total Cholesterol | A | 0.11 | 8 | A < C |
| | B | ns | | |
| Non-HDL Cholesterol | A | 0.03 | 8 | A < C |
| | B | 0.02 | 8 | A < C |
| TC/HDL-C | A | ns | | |
| | B | 0.01 | 8 | A < C |
| Total Testosterone | A | 0.10 | 8 | A > C |
| | B | ns | | |
| Fecal Consistency | A | 0.03 | | A > C |
| | B | 0.08 | | A > C |
| Ease of Stool Passage | A | 0.02 | | A < C |
| | B | 0.09 | | A < C |
| Felt urge to snack? | A | ns | | |
| | B | 0.006 | 2 | A < C |
| Change in Appetite | A | 0.02 | | A < C |
| | B | 0.01 | | A < C |
| Gas | A | <0.05 | | A > C |
| (past 24 hrs) | B | ns | | |
| Abdominal Cramping | A | ns | | |
| (past 24 hrs) | B | ns | | |
| Bloating | A | ns | | |
| (past 24 hrs) | B | ns | | |
| Stomach rumbling | A | <0.05 | | A > C |
| (past 24 hrs) | B | ns | | |

In Table 4 above, directional change of an active group (A) measurement with respect to a control group (C) measurement is indicated. When a statistical calculation applies only to a particular week's measurements in Table 4, the appropriate week is indicated; otherwise, calculations were made over all weeks. Weeks referenced correspond to those previously described and indicated in FIG. 1. The rows marked “ns” were deemed not significant.

The following additional embodiments are provided, the numbering of which is not to be construed as designating levels of importance.

### Additional Embodiments

Embodiment 1 relates to a composition for treating the gastrointestinal (GI) microbiota of a human, the composition comprising: a mixture of microbiota accessible carbohydrates (MACs) comprising; at least two different resistant starches, and at least two different non-starch polysaccharides; and one or more pharmaceutically acceptable additives; wherein the mixture of MACs promotes the activity of amylolytic and short-chain fatty acid (SCFA) producing microbes in the GI system of a human.

Embodiment 2 relates to the composition of Embodiment 1, wherein the resistant starches are selected from the group consisting of starchy vegetables, starch producing roots, tubers, bulbs, corms, legumes, nuts, seeds, and combinations thereof.

Embodiment 3 relates to the composition of Embodiment 1 or 2, wherein the resistant starches are selected from the group consisting of potato starch, pea starch, green plantain starch, tiger nut starch, yacon starch, sweet potato starch, cattail root starch, cattail rhizome starch, cassava starch, arrow root starch, palm starch, chick pea starch, raw green banana starch, and combinations thereof.

Embodiment 4 relates to the composition as set forth in any one of Embodiments 1 to 3, wherein the non-starch polysaccharides are selected from the group consisting of mannans, arabinoglycans, arabinogalactans, rhamnans, beta-glucans, galactans, and combinations thereof.

Embodiment 5 relates to the composition as set forth in any one of Embodiments 1 to 4, wherein each of the at least two different resistant starches are present in the composition in an amount that is at least twice the amount of each of the at least two different non-starch polysaccharides.

Embodiment 6 relates to the composition as set forth in any one of Embodiments 1 to 5, wherein the mixture of MACs promotes amylolytic and SCFA producing activity of at least some members of the taxa belonging to Clostridiaceae, Actinobacteria, and/or Bacteroidetes present in the colon of a human.

Embodiment 7 relates to the composition as set forth in any one of Embodiments 1 to 6, wherein: the at least two different resistant starches comprise pea starch, potato starch, and raw green banana starch; and the at least two different non-starch polysaccharides comprise glucomannon and acacia gum. In a further Embodiment 7a, the at least two different resistant starches further comprises yacon starch.

Embodiment 8 relates to the composition as set forth in any one of Embodiments 1 to 7, wherein the at least two different resistant starches and the at least two different non-starch polysaccharides are present in the mixture of MACs in an amount effective to improve the health of the GI microbiota.

Embodiment 9 relates to the composition of Embodiment 8, wherein improving the health of the GI microbiota includes at least one of increasing a diversity of the GI microbiota, improving a composition of the GI microbiota, and/or increasing SCFA production by the GI microbiota.

Embodiment 10 relates to the composition as set forth in any one of Embodiments 1 to 9, wherein the at least two different resistant starches and the at least two different non-starch polysaccharides are present in the mixture of MACs in an amount effective to improve bowel movement health in a human.

Embodiment 11 relates to the composition as set forth in any one of Embodiments 1 to 10, wherein the at least two different resistant starches and the at least two different non-starch polysaccharides are present in the mixture of MACs in an amount effective to at least one of: increase satiety; promote weight loss; reduce systemic inflammation; increase variance in heart rate; increase skin microbiome diversity in at least one of facial skin and scalp skin; improve facial skin complexion; improve sleep quality; improve blood testosterone levels; improve estradiol blood levels; improve blood lipoprotein lipid levels; improve blood triglyceride levels; improve mood; improve cognition; and/or improve blood cholesterol levels.

Embodiment 12 relates to a blend for inclusion in a dietary supplement, the blend consisting of at least two different resistant starches and at least two different non-starch polysaccharides present in an amount effective to promote the activity of amylolytic and short-chain fatty acid (SCFA) producing microbes in the gastrointestinal (GI) system of a human.

Embodiment 13 relates to the blend of Embodiment 12, wherein each of the at least two different resistant starches is present in an amount greater than each of the at least two different non-starch polysaccharides.

Embodiment 14 relates to the blend of Embodiment 12 or 13, wherein the resistant starches are selected from the group consisting of starchy vegetables, starch producing roots, tubers, bulbs, corms, legumes, nuts, seeds, and combinations thereof.

Embodiment 15 relates to the blend of any one of Embodiments 12 to 14, wherein the resistant starches are selected from the group consisting of potato starch, pea starch, green plantain starch, tiger nut starch, yacon starch, sweet potato starch, cattail root starch, cattail rhizome starch, cassava starch, arrow root starch, palm starch, chick pea starch, raw green banana starch, and combinations thereof.

Embodiment 16 relates to the blend of any one of Embodiments 12 to 15, wherein the non-starch polysaccharides are selected from the group consisting of mannans, arabinoglycans, arabinogalactans, rhamnans, beta-glucans, galactans, and combinations thereof.

Embodiment 17 relates to the blend of any one of Embodiments 12 to 16, wherein the blend promotes the amylolytic and SCFA producing activity of at least some members of the taxa belonging to Clostridiaceae, Actinobacteria, and/or Bacteroidetes present in the colon of a human.

Embodiment 18 relates to the blend of any one of Embodiments 12 to 17, wherein: the at least two different resistant starches comprise pea starch, potato starch, and raw green banana starch; and the at least two different non-starch polysaccharides comprise glucomannon and acacia gum. In a further Embodiment 18a, the at least two different resistant starches further comprises yacon starch.

Embodiment 19 relates to a method of increasing production of short-chain fatty acids in the gastrointestinal (GI) system of a human, the method comprising: orally administering a mixture of microbiota accessible carbohydrates (MACs) comprising at least two different resistant starches and at least two different non-starch polysaccharides; wherein the at least two different resistant starches and the at least two different non-starch polysaccharides are present in the mixture in an amount effective to promote the activity of amylolytic and short-chain fatty acid (SCFA) producing microbes in the GI system of the human.

Embodiment 20 relates to the method of Embodiment 19, wherein the resistant starches are selected from the group consisting of starchy vegetables, starch producing roots, tubers, bulbs, corms, legumes, nuts, seeds, and combinations thereof.

Embodiment 21 relates to the method of Embodiment 19 or 20, wherein the resistant starches are selected from the group consisting of potato starch, pea starch, green plantain starch, tiger nut starch, yacon starch, sweet potato starch, cattail root starch, cattail rhizome starch, cassava starch, arrow root starch, palm starch, chick pea starch, raw green banana starch, and combinations thereof.

Embodiment 22 relates to the method of any one of Embodiments 19 to 21, wherein the non-starch polysaccharides are selected from the group consisting of mannans, arabinoglycans, arabinogalactans, rhamnans, beta-glucans, galactans, and combinations thereof.

Embodiment 23 relates to the method of any one of Embodiments 19 to 22, wherein the at least two different resistant starches and the at least two different non-starch polysaccharides are present in the mixture in an effective amount to promote the amylolytic and SCFA producing activity of at least some members of the taxa belonging to Clostridiaceae, Actinobacteria, and/or Bacteroidetes present in the colon of the human.

Embodiment 24 relates to the method of any one of Embodiments 19 to 23, wherein each of the at least two different resistant starches are present in the mixture in an amount that is at least twice the amount of each of the at least two different non-starch polysaccharides.

Embodiment 25 relates to the method of any one of Embodiments 19 to 24, wherein: the at least two different resistant starches comprise pea starch, potato starch, and raw green banana starch; and the at least two different non-starch polysaccharides comprise glucomannon and acacia gum. In a further Embodiment 25a, the at least two different resistant starches further comprises yacon starch.

Embodiment 26 relates to the method of any one of Embodiments 19 to 25, wherein the at least two different resistant starches and the at least two different non-starch polysaccharides are present in the mixture of MACs in an amount effective to improve the health of the human's GI microbiota.

Embodiment 27 relates to the method of Embodiment 26, wherein improving the health of the GI microbiota includes at least one of increasing a diversity of the GI microbiota, improving a composition of the GI microbiota, and/or increasing SCFA production by the GI microbiota.

Embodiment 28 relates to the method of any one of Embodiments 19 to 27, wherein the at least two different resistant starches and the at least two different non-starch polysaccharides are present in the mixture of MACs in an amount effective to improve bowel movement health in the human.

Embodiment 29 relates to the method of any one of Embodiments 19 to 28, wherein the at least two different resistant starches and the at least two different non-starch polysaccharides are present in the mixture of MACs in an amount effective to at least one of: increase satiety; promote weight loss; reduce systemic inflammation; increase variance in heart rate; increase skin microbiome diversity in at least one of facial skin and scalp skin; improve facial skin complexion; improve sleep quality; improve blood testosterone levels; improve estradiol blood levels; improve blood lipoprotein lipid levels; improve blood triglyceride levels; improve mood; improve cognition; and/or improve blood cholesterol levels.

Embodiment 30 relates to any one of Embodiments 1 to 29, wherein the SCFA producing microbes are selected from the group consisting of acetogenic microbes, butyrogenic microbes, and combinations thereof.

Embodiment 31 relates to use of the composition of any one of Embodiments 1 to 11, use of the blend of any one of Embodiments 12 to 18, or use of the method of any one of Embodiments 19 to 29, for treating the GI microbiota of a human.

Embodiment 32 relates to use of the composition of any one of Embodiments 1 to 11, or use of the blend of any one of Embodiments 12 to 18, for the manufacturing of a product for treating the GI microbiota of a human.

The terms “comprising” or “comprise” are used herein in their broadest sense to mean and encompass the notions of “including”, “include”, “consist(ing) essentially of”, and “consist(ing) of”. The use of “for example”, “e.g.”, “such as”, and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, The term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≥” is “at least” or “greater-than or equal to”; a “<” is “below” or “less-than”; and a “≤” is “at most” or “less-than or equal to”. On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A composition for treating the gastrointestinal (GI) microbiota of a human, the composition comprising:
a mixture of microbiota accessible carbohydrates (MACs) comprising;
at least two different resistant starches, and
at least two different non-starch polysaccharides; and
one or more pharmaceutically acceptable additives;
wherein the mixture of MACs promotes the activity of amylolytic and short-chain fatty acid (SCFA) producing microbes in the GI system of a human.

2. The composition of claim 1, wherein the resistant starches are selected from the group consisting of starchy vegetables, starch producing roots, tubers, bulbs, corms, legumes, nuts, seeds, and combinations thereof.

3. The composition of claim 1, wherein the resistant starches are selected from the group consisting of potato starch, pea starch, green plantain starch, tiger nut starch, yacon starch, sweet potato starch, cattail root starch, cattail rhizome starch, cassava starch, arrow root starch, palm starch, chick pea starch, raw green banana starch, and combinations thereof.

4. The composition of claim 1, wherein the non-starch polysaccharides are selected from the group consisting of mannans, arabinoglycans, arabinogalactans, rhamnans, beta-glucans, galactans, and combinations thereof.

5. The composition of claim 1, wherein each of the at least two different resistant starches are present in the composition in an amount that is at least twice the amount of each of the at least two different non-starch polysaccharides.

6. The composition of claim 1, wherein the mixture of MACs promotes the amylolytic and SCFA producing activity of at least some members of the taxa belonging to Clostridiaceae, Actinobacteria, and/or Bacteroidetes present in the colon of a human.

7. The composition of claim 1, wherein:
the different resistant starches comprise at least three of pea starch, potato starch, raw green banana starch, and yacon starch; and
the different non-starch polysaccharides comprise glucomannon and acacia gum.

8. A blend for inclusion in a dietary supplement, the blend consisting of at least two different resistant starches and at least two different non-starch polysaccharides present in an amount effective to promote the activity of amylolytic and short-chain fatty acid (SCFA) producing microbes in the gastrointestinal (GI) system of a human.

9. The blend of claim 8, wherein each of the at least two different resistant starches is present in an amount greater than each of the at least two different non-starch polysaccharides.

10. The blend of claim 8, wherein the resistant starches are selected from the group consisting of starchy vegetables, starch producing roots, tubers, bulbs, corms, legumes, nuts, seeds, and combinations thereof.

11. The blend of claim 8, wherein the resistant starches are selected from the group consisting of potato starch, pea starch, green plantain starch, tiger nut starch, yacon starch, sweet potato starch, cattail root starch, cattail rhizome starch, cassava starch, arrow root starch, palm starch, chick pea starch, raw green banana starch, and combinations thereof.

12. The blend of claim 8, wherein the non-starch polysaccharides are selected from the group consisting of mannans, arabinoglycans, arabinogalactans, rhamnans, beta-glucans, galactans, and combinations thereof.

13. The blend of claim 8, wherein the blend promotes the amylolytic and SCFA producing activity of at least some members of the taxa belonging to Clostridiaceae, Actinobacteria, and/or Bacteroidetes present in the colon of a human.

14. The blend of claim 8, wherein:
the different resistant starches comprise at least three of pea starch, potato starch, raw green banana starch, and yacon starch; and
the different non-starch polysaccharides comprise glucomannon and acacia gum.

15. A method of increasing production of short-chain fatty acids in the gastrointestinal (GI) system of a human, the method comprising:
orally administering to the human a mixture of microbiota accessible carbohydrates (MACs) comprising at least two different resistant starches and at least two different non-starch polysaccharides;
wherein the different resistant starches and the different non-starch polysaccharides are present in the mixture in an amount effective to promote the activity of amylolytic and short-chain fatty acid (SCFA) producing microbes in the GI system of the human.

16. The method of claim 15, wherein the resistant starches are selected from the group consisting of potato starch, pea starch, green plantain starch, tiger nut starch, yacon starch, sweet potato starch, cattail root starch, cattail rhizome starch, cassava starch, arrow root starch, palm starch, chick pea starch, raw green banana starch, and combinations thereof.

17. The method of claim 15, wherein the non-starch polysaccharides are selected from the group consisting of mannans, arabinoglycans, arabinogalactans, rhamnans, beta-glucans, galactans, and combinations thereof.

18. The method of claim 15, wherein each of the at least two different resistant starches are present in the composition in an amount that is at least twice the amount of each of the at least two different non-starch polysaccharides.

19. The method of claim 15, wherein the mixture of MACs promotes the amylolytic and SCFA producing activity of at least some members of the taxa belonging to Clostridiaceae, Actinobacteria, and/or Bacteroidetes present in the colon of a human.

20. The method of claim 15, wherein:
the different resistant starches comprise at least three of pea starch, potato starch, raw green banana starch, and yacon starch; and
the different non-starch polysaccharides comprise glucomannon and acacia gum.
